(19) United States
(12) Patent Application Publication
(10) Pub. No.: US 2017/0234846 A1
(43) Pub. Date: Aug. 17, 2017

(54) SENSOR DEVICE FOR SENSING FLUORINE-BASED GAS AND METHOD FOR MANUFACTURING THE DEVICE

(71) Applicant: Industry-Academic Cooperation Foundation of Ajou University, Suwon-si (KR)

(72) Inventors: Hyungtak SEO, Seoul (KR); Go Woon SHIM, Suwon-si (KR); Sang Yeon LEE, Suwon-si (KR)

(73) Assignee: Industry-Academic Cooperation Foundation of Ajou University, Suwon-si (KR)

(21) Appl. No.: 15/275,812

(22) Filed: Sep. 26, 2016

(30) Foreign Application Priority Data
Sep. 25, 2015 (KR) 2015-0136194

(51) Int. Cl. G01N 33/00; C25D 11/26; C23C 18/12

(52) U.S. Cl. CPC G01N 33/0052; C23C 18/1208; C25D 11/26

(57) ABSTRACT

In one aspect of the present disclosure, there is provided a sensor device for sensing a fluorine-based gas, the device comprising: a substrate; and a sensing layer on the substrate, wherein the sensing layer includes hydrogenated titanium dioxide nano-particles, wherein when the sensing layer reacts with the fluorine-based gas, the sending layer has a color change.

### CROSS-REFERENCE TO RELATED APPLICATION

This application claims the benefit of Korea patent application No. 10-2015-0136194 filed on Sep. 25, 2015, the entire content of which is incorporated herein by reference for all purposes as if fully set forth herein.

### BACKGROUND

Field of the Present Disclosure

The present disclosure relates to a sensor device for visually and/or electrically sensing a fluorine-based gas and a method for manufacturing the device.

Discussion of Related Art

A fluorine-based gas has been used in various industrial fields including a display, semiconductor, etc. The fluorine-based gas with a low concentration may be very harmful to the human body.

Currently, in order to detect the fluorine, fluoride ions in water are sensed using an electrochemical cell including an electrolyte solution.

Thus, there is a need for a gas sensor to intuitively detect the fluorine-based gas.

### SUMMARY

This Summary is provided to introduce a selection of concepts in a simplified form that are further described below in the Detailed Description. This Summary is not intended to identify all key features or essential features of the claimed subject matter, nor is it intended to be used alone as an aid in determining the scope of the claimed subject matter.

The present disclosure is to provide a sensor device for sensing fluorine-based gas wherein the fluorine-based gas is detected using color-change and electrical conductivity change of a hydrogen-reduced titanium dioxide.

Further, the present disclosure is to provide a method for manufacturing the sensor device for sensing the fluorine-based gas.

In one aspect of the present disclosure, there is provided a sensor device for sensing a fluorine-based gas, the device comprising: a substrate; and a sensing layer on the substrate, wherein the sensing layer includes hydrogenated titanium dioxide nano-particles, wherein when the sensing layer reacts with the fluorine-based gas, the sending layer has a color change.

In one implementation, the hydrogenated titanium dioxide nano-particles include catalyst metal doped therein, wherein the catalyst metal dissociates hydrogen molecules into hydrogen atoms.

In one implementation, the catalyst metal includes at least one selected from a group consisting of palladium (Pd), platinum (Pt), iridium (Ir), rhodium (Rh), sliver (Ag), gold (Au), cobalt (Co), etc.

In one implementation, each of the hydrogenated titanium dioxide nano-particles has a crystalline core and an amorphous shell coating the core.

In one implementation, the device further comprises first and second electrodes on the substrate, wherein the first and second electrodes contact the sensing layer and are spaced from each other; and a measurement unit electrically coupled to the first and second electrodes to measure an electric conductivity change of the sensing layer.

In one aspect of the present disclosure, there is provided a method for manufacturing a sensor device for sensing a fluorine-based gas, the method comprising: mixing a first solution containing a catalyst metal precursor dissolved therein and a second solution containing titanium dioxide nano-particles dispersed therein, to form a mixture solution; irradiating UV-rays to the mixture solution such that the catalyst metal is doped into the titanium dioxide nano-particles; applying thermal treatment to the catalyst metal-doped titanium dioxide nano-particles in a hydrogen gas atmosphere to form hydrogenated catalyst metal-doped titanium dioxide nano-particles; and applying the hydrogenated catalyst metal-doped titanium dioxide nano-particles on a substrate.

In one aspect of the present disclosure, there is provided a method for manufacturing a sensor device for sensing a fluorine-based gas, the method comprising: growing a titanium dioxide nano-structure on a substrate using a hydrothermal method; depositing a catalyst metal on the titanium dioxide nano-structure; and applying thermal treatment to the catalyst metal-deposited titanium dioxide nano-structure in a hydrogen gas atmosphere.

In one aspect of the present disclosure, there is provided a method for manufacturing a sensor device for sensing a fluorine-based gas, the method comprising: anodizing a titanium substrate to form an anodized porous titanium dioxide film in a surface thereof; depositing a catalyst metal on the anodized porous titanium dioxide film; and applying thermal treatment to the catalyst metal-deposited titanium dioxide film in a hydrogen gas atmosphere.

In one implementation of the above-defined methods, the catalyst metal includes at least one selected from a group consisting of palladium (Pd), platinum (Pt), iridium (Ir), rhodium (Rh), sliver (Ag), gold (Au), cobalt (Co), etc.

In accordance with the present disclosure, the present device may reliably, accurately, and intuitively detect the fluorine-based gas via the color-change and electric conductivity change of the sensing layer containing the hydrogenated titanium dioxide nano-particles.

### BRIEF DESCRIPTION OF THE DRAWINGS

The accompanying drawings, which are incorporated in and form a part of this specification and in which like numerals depict like elements, illustrate embodiments of the present disclosure and, together with the description, serve to explain the principles of the disclosure.

FIG. 1 illustrates a structure of a sensor device for sensing a fluorine-based gas in accordance with one embodiment of the present disclosure.

FIG. 2 shows images of a sensing layer including palladium doped titanium dioxide (‘a’), a sensing layer including hydrogenated palladium doped titanium dioxide (‘b’) and a sensing layer including palladium doped titanium dioxide after reaction with XeF₂ gas (‘c’).

FIG. 3 shows HR-TEM images of palladium doped titanium dioxide nano-particle (‘a’, ‘d’), hydrogenated palladium doped titanium dioxide nano-particle (‘b’) and palladium doped titanium dioxide nano-particle (‘c’) after reaction with XeF₂ gas.

FIG. 4 shows current-voltage curves of palladium doped titanium dioxide nano-particle (‘Pd—TiO2’), hydrogenated palladium doped titanium dioxide nano-particle (‘H2 RTA’) and palladium doped titanium dioxide nano-particle after reaction with XeF₂ gas (‘XeF2’).

For simplicity and clarity of illustration, elements in the figures are not necessarily drawn to scale. The same reference numbers in different figures denote the same or similar elements, and as such perform similar functionality. Also, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure.

### DETAILED DESCRIPTIONS

Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

It will be understood that, although the terms “first”, “second”, “third”, and so on may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present disclosure.

It will be understood that when an element or layer is referred to as being “connected to”, or “coupled to” another element or layer, it can be directly on, connected to, or coupled to the other element or layer, or one or more intervening elements or layers may be present. In addition, it will also be understood that when an element or layer is referred to as being “between” two elements or layers, it can be the only element or layer between the two elements or layers, or one or more intervening elements or layers may also be present.

Spatially relative terms, such as “beneath,” “below,” “lower,” “under,” “above,” “upper,” and the like, may be used herein for ease of explanation to describe one element or feature's relationship to another element s or feature s as illustrated in the figures. It will be understood that the spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented for example, rotated 90 degrees or at other orientations, and the spatially relative descriptors used herein should be interpreted accordingly.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, s, operations, elements, and/or components, but do not preclude the presence or addition of one or more other features, integers, s, operations, elements, components, and/or portions thereof. As used herein, the term “and/or” includes any and all combinations of one or more of the associated listed items. Expression such as “at least one of” when preceding a list of elements may modify the entire list of elements and may not modify the individual elements of the list.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In the following description, numerous specific details are set forth in order to provide a thorough understanding of the present disclosure. The present disclosure may be practiced without some or all of these specific details. In other instances, well-known process structures and/or processes have not been described in detail in order not to unnecessarily obscure the present disclosure.

As used herein, the term “substantially,” “about,” and similar terms are used as terms of approximation and not as terms of degree, and are intended to account for the inherent deviations in measured or calculated values that would be recognized by those of ordinary skill in the art. Further, the use of “may” when describing embodiments of the present disclosure refers to “one or more embodiments of the present disclosure.”

FIG. 1 illustrates a structure of a sensor device for sensing a fluorine-based gas in accordance with one embodiment of the present disclosure.

Referring to FIG. 1, a sensor device 100 for sensing a fluorine-based gas in accordance with one embodiment of the present disclosure may include a substrate 110 and a sensing layer 120 thereon. The fluorine-based gas may refer to a gas containing fluorine elements. Examples thereof may include, by way of example, a xenon fluoride gas such as XeF₂, XeF₄, XeF₆, etc., a carbon fluoride gas such as CF₄, etc., a sulfur fluoride gas such as SF₆, etc.

The substrate 110 may have various structures may be made of various materials. For example, the substrate 110 may be made of a paper, polymer, ceramic, glass, metal, etc.

The sensing layer 120 may be disposed on the substrate 110. The sensing layer 120 may be configured to detect the fluorine-based gas via color-change and/or electric conductivity change.

In one embodiment, the sensing layer 120 may include nano-particles of a hydrogenated titanium dioxide. In the present disclosure, a term ‘hydrogenated titanium dioxide’ may refer to a hydrogen-doped titanium dioxide, wherein a doped hydrogen ion may be bonded to an oxygen ion and/or titanium ion in the titanium dioxide. In the present disclosure, a term ‘nano-particle’ may include not only a 3-dimensional nano-powder particle with an average diameter in a range of several nanometers to several hundred nanometers but also a liner nano-rod with an average diameter in a range of several nanometers to several hundred nanometers.

A non-hydrogenated titanium dioxide may not absorb a visible ray and, thus, render a white or colorlessness. The hydrogenated titanium dioxide may have a Fermi energy level and, thus, may absorb a visible ray to render a black or dark gray. Further, when the hydrogenated titanium dioxide reacts with the fluorine-based gas, the hydrogen ion in the hydrogenated titanium dioxide is substituted with the fluorine ion, thereby to raise the Fermi energy level, and, thus, to render a light gray or white. The sensing layer 120 may sense the fluorine-based gas using the above-described color-change of the hydrogenated titanium dioxide nano-particles.

With reference to a following reaction expression 1, a reaction between hydrogenated titanium dioxide nano-particles of the sensing layer 120 and a fluorine-based gas (XFₓ(g)) will be described:

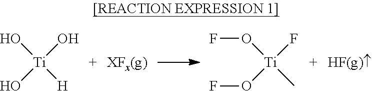

As shown in the reaction expression 1, when the hydrogenated titanium dioxide contacts with the fluorine-based gas, some of fluorine ions with high electronegativity may invade into hydrogen ion sites and be bonded to the oxygen ion and/or titanium ion; the other of the fluorine ions may react with the hydrogen ions to produce a hydrogen fluoride gas. As described above, via the above reaction, the color of the hydrogenated titanium dioxide may change from the black or dark gray to the light gray or white.

In one embodiment, the hydrogenated titanium dioxide nano-particles may contain a doped catalyst metal therein. For example, in order to facilitate hydrogenation of the titanium dioxide nano-powders, the hydrogenated titanium dioxide nano-particles may have a doped catalyst metal therein capable of dissociating hydrogen molecules into hydrogen atoms. The doped catalyst metal therein capable of dissociating hydrogen molecules into hydrogen atoms may include, by way of example, one selected from a group consisting of palladium (Pd), platinum (Pt), iridium (Ir), rhodium (Rh), sliver (Ag), gold (Au), cobalt (Co), etc.

The hydrogenated titanium dioxide nano-particles with the above-defined doped catalyst metal therein may have a crystalline core and an amorphous shell coating the core. The crystalline core may have an anatase or rutile crystalline phase.

The sensing layer 120 may be formed on the substrate 110 using various methods.

In one embodiment, the sensing layer 120 may be formed by mixing between a solution containing a catalyst metal ion and a titanium dioxide nano-particle dispersed solution, and performing UV-rays irradiation to the mixture to allow the catalyst metal to be doped into the titanium dioxide nano-particle, and applying thermal treatment to the resulting mixture in a

hydrogen gas atmosphere to form the hydrogenated titanium dioxide nano-particle with the doped catalyst metal, and applying the hydrogenated titanium dioxide nano-particle on the substrate 110.

In another embodiment, the sensing layer 120 be formed by growing a titanium dioxide nano-structure on the substrate 110 using a hydrothermal method, and depositing the catalyst metal thereon, and, then applying thermal treatment thereto in a hydrogen gas atmosphere.

In still another embodiment, the sensing layer 120 may be formed by anodizing a titanium substrate 110 to form a porous titanium dioxide film in a surface thereof, depositing the catalyst metal thereon, and, then applying thermal treatment thereto in a hydrogen gas atmosphere.

The sensor device 100 for sensing the fluorine-based gas may further include first and second electrodes 130A, 130B on the substrate 110, wherein the first and second electrodes 130A, 130B contact the sensing layer 120 and are spaced from each other; and a measurement unit 140 electrically coupled to the first and second electrodes 130A, 130B to measure the electric conductivity change of the sensing layer 120.

When the hydrogenated titanium dioxide nano-particles of the sensing layer 120 contact the fluorine-based gas, the electrical conductivity may increase. In this way, in the present disclosure, the measurement unit 140 may detect the fluorine-based gas by measuring the electric conductivity change of the sensing layer 120.

The sensor device 100 for sensing the fluorine-based gas 100 may accurately and rapidly and intuitively sense the fluorine-based gas using the color-change and electric conductivity change of the sensing layer 120 having the hydrogenated titanium dioxide nano-particles.

Hereinafter, specific embodiments of the present disclosure will be described. The following specific embodiments of the present disclosure may be merely examples of the present disclosure. Thus, the present disclosure may not be limited to the following embodiments.

### Embodiment

18 mg PdCl₂ is added into 100 ml methanol in a first container to produce a mixture, which, in turn, is subjected to an ultrasonic wave for 2 to 3 hours. Then, 25 mg PVP (Polyvinyl pyrrolidone) is added into the mixture and is agitated for 10 hours to form a first solution. Titanium dioxide nano-particle 1 g with an average diameter of 30 nm are added into 100 ml methanol in a second container to produce a mixture, which, in turn, is subjected to an ultrasonic wave, to form a second solution.

Then, the first solution and second solution are mixed and are agitated for 2 to 3 hours to form a mixture which is subjected to UV-rays irradiation for 2 mins to allow palladium (Pd) to be doped into the titanium dioxide nano-particle.

Thereafter, the doped titanium dioxide particles with the doped palladium (Pd) are collected by a centrifugation method and are dried and are dispersed in ethanol solvent. Then, the dispersion is applied to a glass fiber filter paper and is dried in an oven at 60° C.

Subsequently, the glass fiber filter paper with the doped titanium dioxide particles with the doped palladium thereon is subjected to thermal treatment in a H₂/N₂ 5% gas atmosphere at 400° C. for 5 mins, thereby to form a sensing layer made of the hydrogenated palladium-doped titanium dioxide nano-particles on the glass fiber filter paper.

### Example

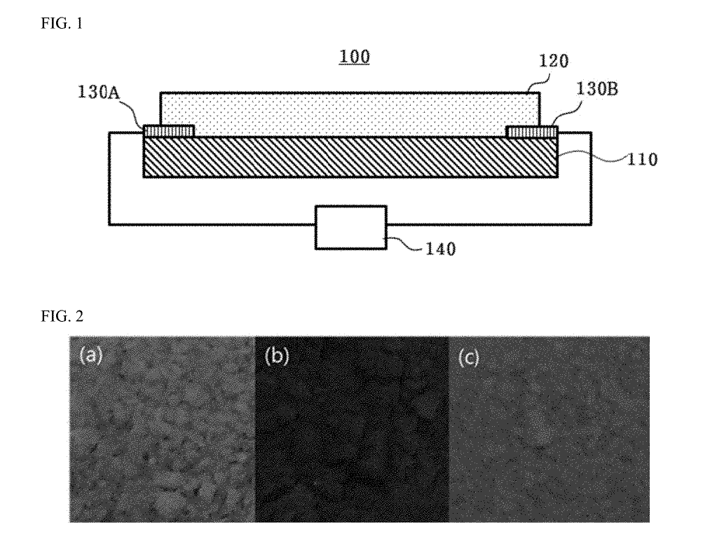

FIG. 2 shows images of a sensing layer including palladium doped titanium dioxide (‘a’), a sensing layer including hydrogenated palladium doped titanium dioxide (‘b’) and a sensing layer including palladium doped titanium dioxide after reaction with XeF₂ gas (‘c’).

Referring to FIG. 2, the sensing layer including hydrogenated palladium doped titanium dioxide (‘b’) renders a black or dark gray, while the sensing layer including palladium doped titanium dioxide after reaction with XeF₂ gas (‘c’) renders a light gray.

In this way, the present sensor device may detect the fluorine-based gas via the color-change of the hydrogenated palladium doped titanium dioxide (‘b’).

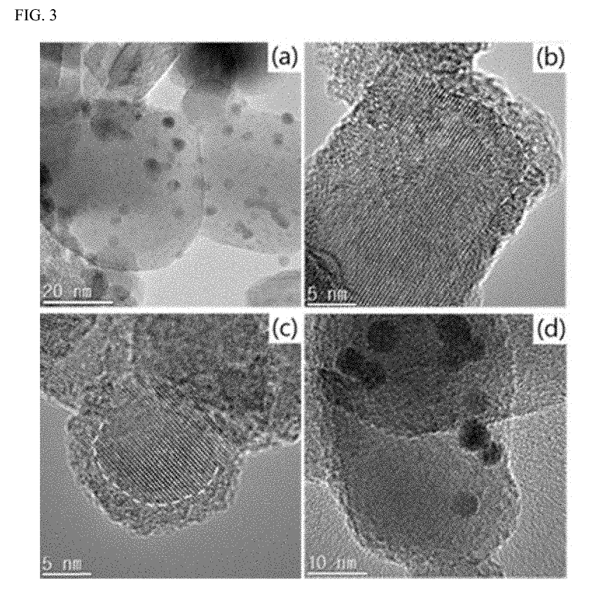

FIG. 3 shows HR-TEM images of palladium doped titanium dioxide nano-particle (‘a’, ‘d’), hydrogenated palladium doped titanium dioxide nano-particle (‘b’) and palladium doped titanium dioxide nano-particle (‘c’) after reaction with XeF₂ gas.

Referring to FIG. 3, the palladium doped titanium dioxide nano-particle has

a crystalline structure in an entirety thereof, while the hydrogenated palladium doped titanium dioxide nano-particle has a crystalline structure at an inner portion and has an amorphous film with about 3 nm thickness in a surface thereof due to the reaction with the hydrogen.

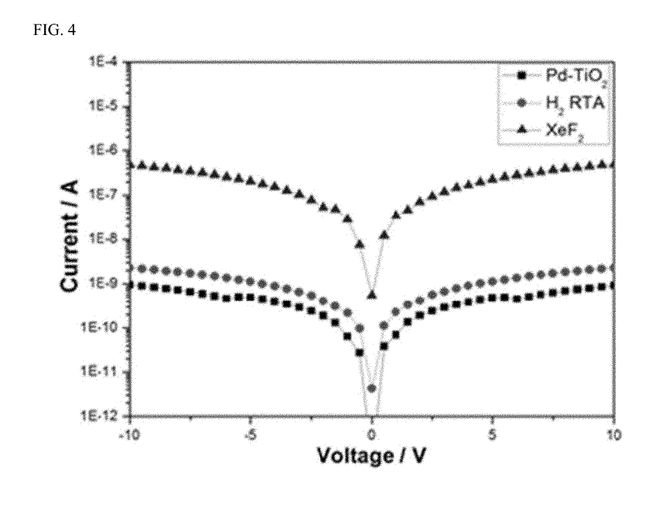

FIG. 4 shows current-voltage curves of palladium doped titanium dioxide nano-particle (‘Pd—TiO2’), hydrogenated palladium doped titanium dioxide nano-particle (‘H2 RTA’) and palladium doped titanium dioxide nano-particle after reaction with XeF₂ gas (‘XeF2’).

Referring to FIG. 4, when the hydrogenated palladium doped titanium dioxide nano-particle reacts with the XeF₂ gas, the current may increase at least about 100 times. This is because a fluorine (F) introduced into the titanium dioxide may act as a n-type donor, thereby to increase the Fermi energy level E_{F} nearby to a conduction band. Thus, when the hydrogenated palladium doped titanium dioxide nano-particle is contained in the sensing layer, the present device may detect the fluorine-based gas via the electric conductivity change of the sensing layer.

The above description is not to be taken in a limiting sense, but is made merely for the purpose of describing the general principles of exemplary embodiments, and many additional embodiments of this disclosure are possible. It is understood that no limitation of the scope of the disclosure is thereby intended. The scope of the disclosure should be determined with reference to the Claims. Reference throughout this specification to “one embodiment,” “an embodiment,” or similar language means that a particular feature, structure, or characteristic that is described in connection with the embodiment is included in at least one embodiment of the present disclosure. Thus, appearances of the phrases “in one embodiment,” “in an embodiment,” and similar language throughout this specification may, but do not necessarily, all refer to the same embodiment.

1. A sensor device for sensing a fluorine-based gas, the device comprising:
a substrate; and
a sensing layer on the substrate, the sensing layer comprising hydrogenated titanium dioxide nano-particles of which a color is changed by a reaction with the fluorine-based gas.

2. The device of claim 1, wherein the hydrogenated titanium dioxide nano-particles comprises catalyst metal doped therein, wherein the catalyst metal dissociates hydrogen molecules into hydrogen atoms.

3. The device of claim 2, wherein the catalyst metal comprises at least one selected from a group consisting of palladium (Pd), platinum (Pt), iridium (Ir), rhodium (Rh), sliver (Ag), gold (Au), and cobalt (Co).

4. The device of claim 1, wherein each of the hydrogenated titanium dioxide nano-particles has a crystalline core and an amorphous shell on a surface of the core.

5. The device of claim 1, further comprising:
first and second electrodes on the substrate, wherein the first and second electrodes contact the sensing layer and are spaced apart from each other; and
a measurement unit electrically coupled to the first and second electrodes to measure an electric conductivity change of the sensing layer.

6. A method for manufacturing a sensor device for sensing a fluorine-based gas, the method comprising:
mixing a first solution containing a catalyst metal precursor dissolved therein and a second solution containing titanium dioxide nano-particles dispersed therein, to form a mixture solution;
irradiating UV-rays to the mixture solution such that the catalyst metal is doped into the titanium dioxide nano-particles;
applying thermal treatment to the catalyst metal-doped titanium dioxide nano-particles in a hydrogen gas atmosphere to form hydrogenated catalyst metal-doped titanium dioxide nano-particles; and
applying the hydrogenated catalyst metal-doped titanium dioxide nano-particles on a substrate.

7. A method for manufacturing a sensor device for sensing a fluorine-based gas, the method comprising:
growing a titanium dioxide nano-structure on a substrate using a hydrothermal method;
depositing a catalyst metal on the titanium dioxide nano-structure; and
applying thermal treatment to the catalyst metal-deposited titanium dioxide nano-structure in a hydrogen gas atmosphere.

8. A method for manufacturing a sensor device for sensing a fluorine-based gas, the method comprising:
anodizing a titanium substrate to form an anodized porous titanium dioxide film in a surface thereof;
depositing a catalyst metal on the anodized porous titanium dioxide film; and
applying thermal treatment to the catalyst metal-deposited titanium dioxide film in a hydrogen gas atmosphere.

9. The method of claim 6, wherein the catalyst metal includes at least one selected from a group consisting of palladium (Pd), platinum (Pt), iridium (Ir), rhodium (Rh), sliver (Ag), gold (Au), and cobalt (Co).

10. The method of claim 7, wherein the catalyst metal includes at least one selected from a group consisting of palladium (Pd), platinum (Pt), iridium (Ir), rhodium (Rh), sliver (Ag), gold (Au), and cobalt (Co).

11. The method of claim 8, wherein the catalyst metal includes at least one selected from a group consisting of palladium (Pd), platinum (Pt), iridium (Ir), rhodium (Rh), sliver (Ag), gold (Au), and cobalt (Co).
